(19) United States
(12) Patent Application Publication
(10) Pub. No.: US 2006/0015144 A1
(43) Pub. Date: Jan. 19, 2006

(54) Uterine artery occlusion staple

(75) Inventors: Fred H. Burbank, Laguna Niguel, CA (US); Michael L. Jones, San Clemente, CA (US); Greig E. Altieri, Laguna Beach, CA (US); Jill Uyeno, Mission Viejo, CA (US)

Correspondence Address: Edward J. Lynch;DUANE MORRIS LLP, One Market, Spear Tower, Suite 2000, San Francisco, CA, 94105, US

(73) Assignee: Vascular Control Systems, Inc.

(21) Appl. No.: 10/894,886

(22) Filed: Jul. 19, 2004

(51) Int. Cl. A61B 17/08

(52) U.S. Cl. 606/219

(57) ABSTRACT

The invention is directed to an intravaginal staple for occluding a female patient's uterine artery in the treatment of a uterine disorder such as fibroids, DUB, PPH, uterine bleeding after caesarian delivery and the like. The intravaginal staple includes an occluding bar having a pressure applying surface and at least one and preferably two tissue penetrating legs which are provided with protuberances which help to retain the staple leg within the penetrated tissue. At least part of the staple is preferably formed of bioabsorbable material so that it will in part self destruct at a preselected time period by separation of staple components caused by the bioabsorption of a bioabsorbable portion of the staple. The staple deployment system may be provided with elongated handles and staple deploying mechanisms on the distal ends of the handles to drive the legs of the staple into the tissue bundle about the uterine artery so that pressure applied by the occlusion bar occludes the uterine artery.

### FIELD OF THE INVENTION

The invention is generally directed to the treatment of uterine disorders by detecting and regulating blood flow through one or both of the patient's uterine arteries.

### BACKGROUND OF THE INVENTION

Hysterectomy (surgical removal of the uterus) is performed on approximately 600,000 women annually in the United States. Hysterectomy is often the therapeutic choice for the treatment of uterine cancer, adenomyosis, menorrhagia, prolapse, dysfunctional uterine bleeding (abnormal menstrual bleeding that has no discrete anatomic explanation such as a tumor or growth), and muscular tumors of the uterus, known as leimyoma or uterine fibroids.

However, hysterectomy is a drastic treatment, having many undesirable characteristics. Thus, any method which can approximate the therapeutic result of a hysterectomy without removing the uterus would be a significant improvement in this field. Newer treatment methods have been developed for some diseases which may spare these women a hysterectomy.

In 1995, it was demonstrated that uterine fibroids could be treated without hysterectomy using a non-surgical therapy, specifically comprising bilateral intraluminal occlusion of the uterine arteries (Ravina et al., “Arterial Embolization to Treat Uterine Myomata”, Lancet Sep. 9, 1995; Vol. 346; pp. 671-672, incorporated in its entirety herein). This technique is known as “uterine artery embolization”. In this technique, uterine arteries are accessed via a transvascular route from a common femoral artery into the left and right uterine arteries by means of an intravascular catheter and embolic material, such as small metallic coils, polyvinyl alcohol particulate and the like, is delivered through the catheter to the uterine arteries which quickly become occluded.

The uterus has a dual (or redundant) blood supply, the primary blood supply being from the bilateral uterine arteries, and the secondary blood supply from the bilateral ovarian arteries. Consequently, when both uterine arteries are occluded, i.e. bilateral vessel occlusion, the uterus and the fibroids contained within the uterus are both deprived of their blood supply. However, as demonstrated by Ravina et al., the ischemic effects on the fibroid is greater than the effect on the uterus. In most instances, the fibroid withers and ceases to cause clinical symptoms.

However, many physicians do not possess the training or equipment necessary to perform catheter-based uterine artery embolization under radiologic direction. Accordingly, there are substantially fewer uterine artery embolizations performed, worldwide, each year than hysterectomies for symptomatic uterine fibroids.

Recently, fibroid treatment procedures have been described wherein the uterine arteries are temporarily occluded by an intravaginal device which is non-invasively pressed against the patient's vaginal fornix and clamped or otherwise pressed against tissue bundle with the patient's uterine artery being within the bundle. Pressure on the tissue occludes the underlying uterine artery. While these procedures have shown much promise the clamps were for the most part required to be left in the patient which restricted the patient to a hospital or a clinical setting for the duration of the treatment.

What is needed, therefore, are devices and methods to detect blood vessels and blood flow in blood vessels, and devices and methods to occlude blood flow in blood vessels such as the uterine arteries that can be used by physicians with limited training and equipment and allows the patient to quickly return to normal activity.

### SUMMARY OF THE INVENTION

The invention is directed to an intra-vaginal staple for occluding a female patient's uterine artery by means of a relatively non-invasive intravaginal delivery device and to a system and a procedure for installing the staple.

An intravaginal staple embodying features of the invention includes an occlusion bar which has a pressure applying surface configured to apply pressure against the exterior of the patient's uterine cervix or against the patient's vaginal fornix with sufficient pressure to occlude an underlying uterine artery. The intravaginal staple also has at least one leg which extends distally away from the pressure applying surface of the occlusion bar and which is configured to penetrate through the wall of the patient's vaginal fornix until at least the distal portion of the leg or legs advance into the patient's uterine wall or other tissue. The leg or legs are configured to secure the staple with the occlusion bar applying sufficient pressure against the patient's vaginal fornix to ensure at least partial occlusion of an underlying uterine artery.

At least part of the intravaginal staple should be formed of bioabsorbable material so that the occlusion bar occludes the uterine artery for a selected time period for the therapeutic affects desired and then the bioabsorbable portion of the staple breaks down which in turn releases the pressure applied by the occlusion bar. The staple legs, the protrusions on one or more of the staple legs, the occlusion bar, or the junction between the legs and the occlusion bar may be formed of suitable bioabsorbable material which is absorbed at a suitable rate for the desired break down of the staple.

In one embodiment of the invention, the staple or the staple delivery system is provided with a blood flow sensor is be applied to a portion of a vaginal wall to detect and/or locate and/or monitor the occlusion of the underlying uterine artery. The wall of the patient's vaginal fornix may be distended by an staple deployment so as to more closely approach the underlying uterine artery. Such an approach may aided by applying tension to the uterus e.g., by pulling on the uterine cervix by any suitable device or implement such as a tenaculum. However, other instruments such as forceps, suction devices, and the like may be employed to pull on the uterine cervix.

A suitable staple delivery or deployment system embodying features of the invention may include an elongated handle having an operative proximal end configured to extend out of a patient to facilitate manual manipulation and an operating distal end which has a staple delivery mechanism which is configured to engaged and apply a staple assembly to the patient's uterine artery. The staple delivery mechanism has a recess for receiving a staple or staple assembly and a driving member or piston in the recess configured to drive the staple or staple assembly into the patient's tissue. The piston may be driven by mechanical, hydraulic or electromechanical means.

Preferably, the staple deployment system is a clamp-type instrument having a pair of elongated handles, with a staple deployment mechanism as described above on each distal end of the handles and a finger grip at each proximal end of the handles. The handles are preferably pivotally connected to facilitate adjustment of the spacing between the staple deployment mechanisms on the distal ends for proper placement of the staples when they are deployed. The handles are configured so the finger grips on the proximal ends of the handles extend out of the patient when the staple deployment mechanism is in position for staple deployment.

An alternative staple assembly includes a generally U-shaped staple member with an occlusion bar having a bore at each end which slidably received the legs of the staple member. The pressure applying surface of the occlusion bar may be provided with a blood flow sensor to ensure proper placement of the bar during deployment.

The staple deployment mechanism or the staple assembly itself is preferably provided with a blood flow sensor for locating the uterine artery to be occluded and monitoring blood flow through the artery after it has been occluded by the staple. The sensor may sense sound, pressure, strain, stress, chemical entity, electromagnetic radiation and the like, and may be a combination of such sensors. A preferred sensor is a Doppler ultrasound sensor. The blood flow sensor is preferably mounted to the face of a tissue-contacting surface of the staple deployment mechanism, such as adjacent to the recess which receives the staple. The sensor should be positioned between the legs of the staple so that the located uterine artery is centrally disposed between the staple legs when the staple is deployed. This ensures that the legs do not penetrate into the arterial wall. Ultrasound energy useful for sensing a location of a blood vessel or of blood flow in a blood vessel has a frequency of less than about 20 MegaHertz (MHz), such as between about 5 MHz and about 19 MHz, and preferably between about 6 MHz and about 10 MHz. In commercially available Doppler sensors the frequency is typically about 8 MHz. For sensors based on electromagnetic (EM) energy useful for sensing a location of a blood vessel or of blood flow in a blood vessel, the EM energy should have a wavelength of about 500 nanometers (nm) to about 2000 nm, preferably about 700 nm to about 1000 nm.

A method for occluding a uterine artery which embodies features of the invention include advancing the staple deployment system through the patient's vaginal canal until each of the staple deployment mechanisms are on adjacent sides of the patient's uterine cervix. The leading edges of the deployment mechanisms are pressed against the wall of the vaginal fornix on each side of the cervical os and the spacing therebetween is adjusted to ensure the staple will be deployed at the proper location. Adjustment of the positions of the staple deployment mechanisms on the distal ends of the handles allows the blood flow sensors on the mechanisms to locate the uterine artery which is a short distance from the surface of the vaginal fornix. With the deployment mechanisms adjacent to the target blood vessels, the leading edges of the deployment mechanisms can be pressed against the patient's vaginal fornix to compress tissue thereof and thereby occlude the underlying uterine arteries. The uterine arteries are located with the blood flow sensors on the leading edges of the staple deployment mechanism. Preferably, tension is applied to the uterine cervix with a grasping implement, such as a tenaculum, (e.g., by pulling on the uterine cervix) while applying force or pressure to a vaginal wall to occlude a uterine artery.

The invention allows for the non-surgical location and occlusion of blood vessels such as the uterine artery, providing effective therapeutic treatment. Importantly, the present invention allows for the occlusion of a female patient's uterine artery without the need for radiographic equipment or for extensive training in the use of radiographic techniques. The devices and methods are simple and readily used for treating uterine fibroids, dysfunctional uterine bleeding (DUB), adenomyosis, post-partum hemorrhage, and other uterine disorders or conditions which may be treated by uterine artery occlusion.

These and other advantages will become more apparent from the following detailed description when taken in conjunction with the accompanying exemplary drawings.

### BRIEF DESCRIPTION OF THE DRAWINGS

FIG. 1 is an elevational view of a uterine artery staple embodying features of the invention.

FIGS. 2a-2d are various transverse cross-sections taken along the lines 2-2 in FIG. 1

FIGS. 3a-3d are various transverse cross-sections taken along the lines 3-3 in FIG. 1.

FIG. 4 is a perspective view of a staple delivery device for the staple shown in FIG. 1.

FIG. 5 is a plan view of the staple delivery device shown in FIG. 4.

FIG. 6 is an end view of the staple delivery device shown in FIG. 4.

FIG. 7 is a side view of the staple delivery device shown in FIG. 4.

FIG. 8 is a schematic, elevational view of a staple deployment mechanism suitable for use with the staple delivery device shown in FIG. 4

FIG. 9 is a top view of the staple deployment mechanism shown in FIG. 8.

FIG. 10 is a side view of the staple deployment mechanism shown in FIG. 8.

FIG. 11 is an elevational view of the staple deployment mechanism shown in FIG. 8 with the staple fully deployed.

FIG. 12 is a perspective view of the staple deployment mechanism shown in FIG. 11 with the staple fully deployed.

FIG. 13 is an elevational view of the staple deployment mechanism shown in FIG. 11 with the staple deployed to occlude the patient's uterine artery.

FIG. 14 is an elevational view, partially in section, of an alternative staple assembly which includes a slidable pressure applying occlusion bar with a blood flow sensor and a U-shaped staple member.

FIG. 15 is a plan view, partially in section, taken along the lines 15-15 in FIG. 14.

### DETAILED DESCRIPTION OF THE INVENTION

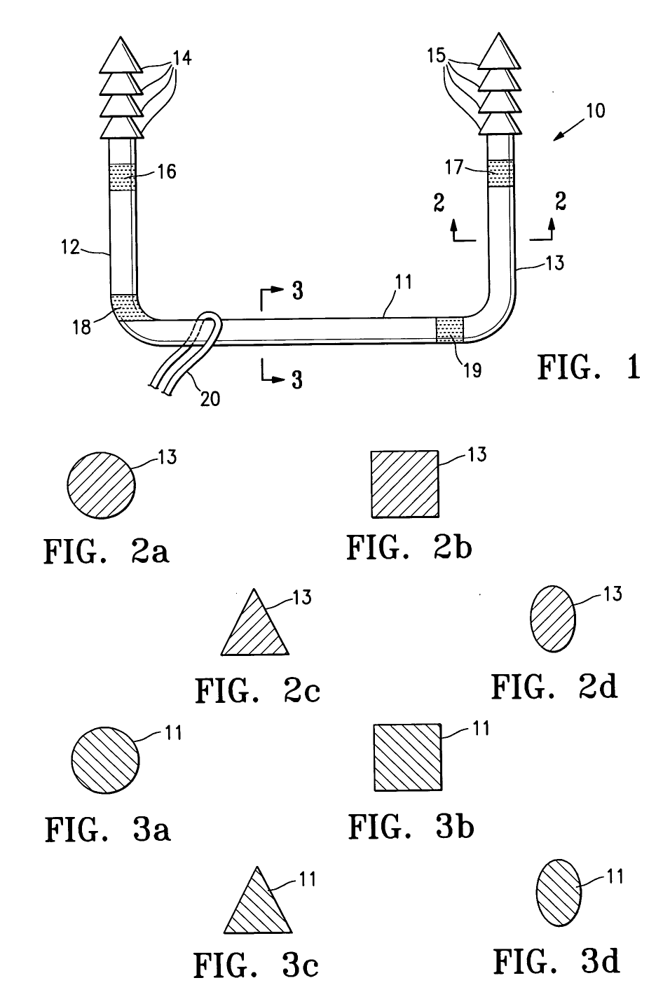

FIG. 1 illustrates a uterine artery staple 10 which has a pressure applying occlusion bar 11 which extends between two tissue penetrating legs 12 and 13. The legs 12 and 13 are provided with a plurality of protuberances or barbs 14 and 15 respectively which help to retain the legs of staple 10 in tissue after placement therein. At least part of the staple 10, illustrated at locations 16-19, is formed of a bioabsorbable material such as polylactic acid, polyglycolic acid or copolymers or blends thereof. When the staple 10 is deployed into the patient to occlude the patient's uterine artery, the occlusion bar 11 of the staple is retained for a prescribed length of time in a pressure applying condition against the patient's uterine artery for the occlusion thereof. After the prescribed length of time, the bioabsorbable material at one or more of the locations is absorbed, separating the retained portion of the staple, e.g. the legs from the pressure applying portion of the staple, e.g. the occlusion bar 11, thereby releasing the pressure applied by the bar 11 to occlude the uterine artery. Upon release of the occluding pressure, blood flow may resume through the patient's uterine artery. Alternatively, the protuberances 14 and 15 may be formed of bioabsorbable material, which when the material thereof is bio-absorbed, the entire staple may be easily removed. A strand 20 may be connected to the occlusion bar 11 to facilitate removal of the occlusion in such an instance. Once the bioabsorption has taken place, a tug may be applied to the strand 20 to pull on the occlusion bar 11 or the entire staple out of the tissue. The end of the strand 20 may extend to an accessible location within the patient's vaginal canal or may extend outside the patient through the vaginal opening.

The occlusion bar 11 and the legs 12 and 13 may have a variety of transverse cross-sectional shapes as illustrated in FIGS. 2a-2d for the legs 12 and 13 and in FIGS. 3a-3d for the occlusion bar 11. Moreover, the legs 12 and 13 need not be of the same length, at the same angle with respect to the occlusion bar 11 or each other.

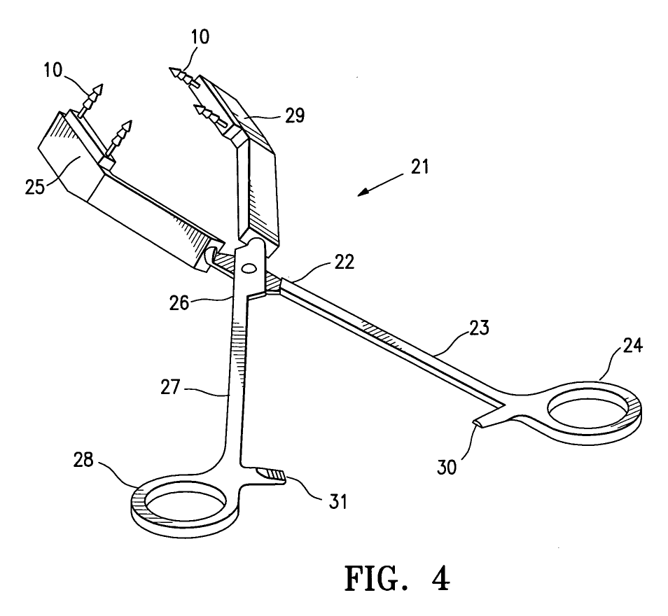

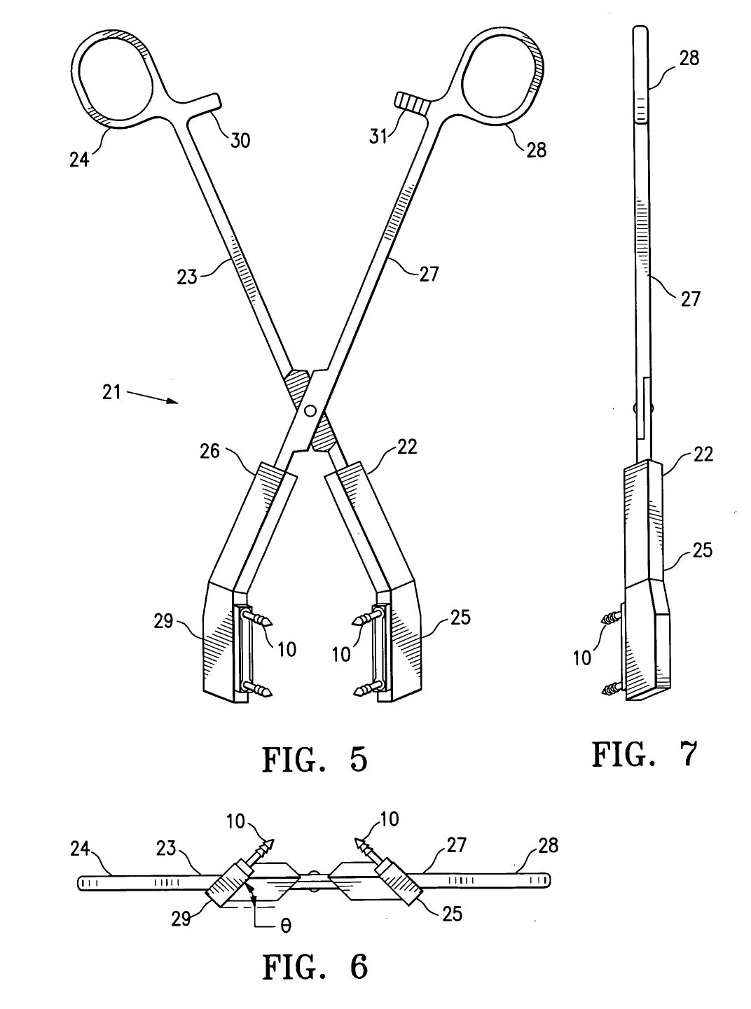

FIG. 4-7 depict a relatively non-invasive, intravaginal staple delivery system 21 embodying features of the invention. The staple delivery system 21 includes a first elongated staple delivery member 22 having an elongated handle 23 with a finger grip 24 at the proximal end, and a staple deployment mechanism 25 on the distal end of the handle 23 at an appropriate angle with respect to the longitudinal axis of the handle to facilitate placement against the patient's vaginal fornix and the appropriate deployment of staple 10. A second elongated staple delivery member 26, which is essentially the mirror image of the staple delivery member 22, has an elongated handle 27 with a finger grip 28 at the proximal end and a staple deployment mechanism 29 on the distal end. Each of the handles 23 and 27 are provided with a ratchet member 30 and 31 respectively which interact to lock the relative positions of the staple deployment mechanisms 25 and 29 on the distal ends of the handles 23 and 27 respectively. The ratchet members 30 and 31 are preferably releasable so that the staple deploying mechanisms 25 and 29 can be released after the staples have been deployed to occlude the patient's uterine arteries and the staple delivery system 10 removed from the patient.

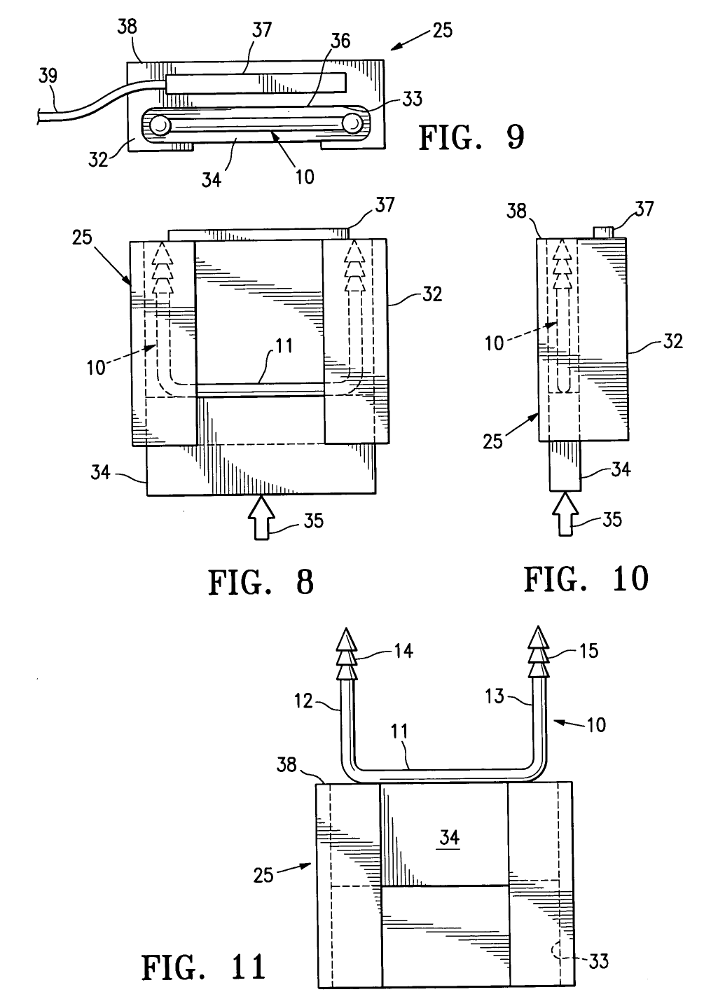

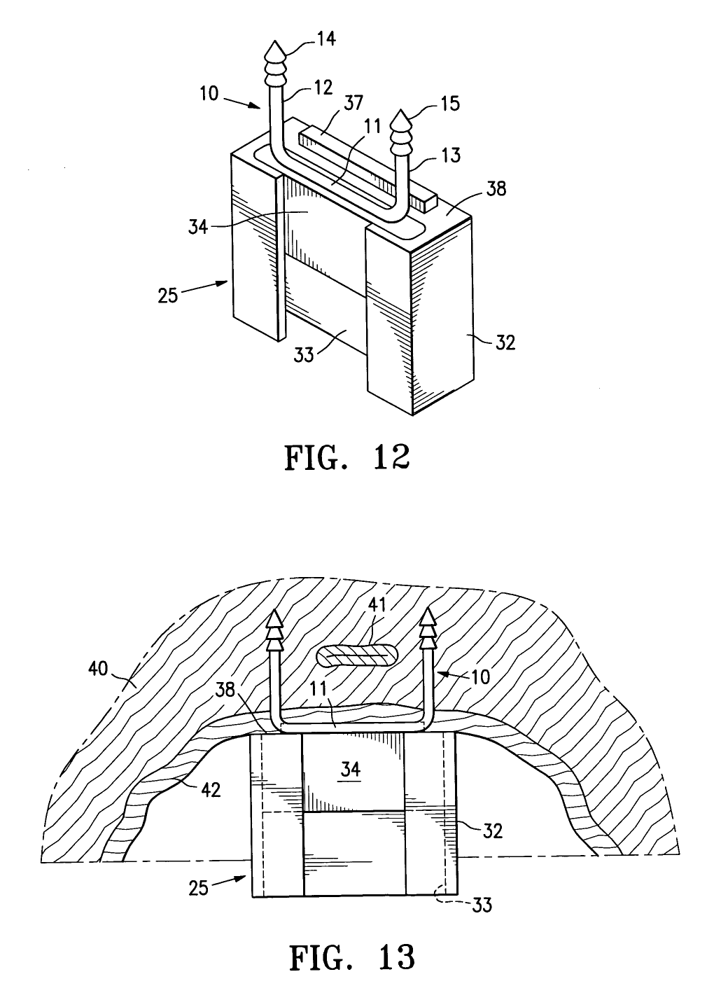

The staple delivery mechanism 25, which is shown schematically in more detail in FIGS. 8-12, includes a staple receptor 32 having a slot or recess 33 configured to slidably receive a staple 10. A staple driver 34 is provided at the lower portion of the slot 33 to drive the staple 10 out of the slot 32 of the staple receptor 31 with sufficient force to ensure that the legs 12 and 13 of the staple 10 penetrate well into the patient's tissue and the occluding bar 11 presses against the exterior of the tissue so as to occlude an underlying uterine artery. The protuberances 14 and 15 on legs 12 and 13 respectively retain the staple 10 within the penetrated tissue for the prescribed length of time to ensure adequate occlusion of the patient's uterine artery. The staple driver is depicted as being driven by the arrow 35, which may be hydraulically, mechanically or electromechanically driven, to eject the staple 10 out of the staple receiving slot or recess 33 and into the patient's tissue which may surround the slot opening 36. Deployment of the staple 10 is shown in FIGS. 11 and 12.

The staple receptor 33 is provided with a blood flow sensor 37 on the upper or leading surface 38 adjacent to the slot opening 36 to facilitate location of the patient's uterine artery as the staple delivery mechanism 25 is pressed against the patient's vaginal fornix during staple deployment. The preferred blood flow sensor 37 is a Doppler ultrasonic fluid flow sensing system. The sensor location on the leading surface 38 allows the operator to more easily guide the staple delivery system 21 to an appropriate location of the patient's target uterine artery. Sensor 36 is provided with a signal and/or energy transmission cable 39 which is operatively connected to sensor control device (not shown). Cable 39 may be an insulated wire, plurality of wires, optical fiber, waveguide, or other connection effective to carry signals and/or energy or power to and/or from the sensor 36.

Blood flow sensor 36 may be a passive sensor which is configured to detect intrinsic signals indicating the presence of a blood vessel (i.e., a sound sensor, a motion sensor, a pH sensor, or other sensor configured to detect a physical, chemical, electrical, or physiological indication of the location of a blood vessel). In other embodiments, the blood flow sensor 36 may be an active sensor, configured to emit energy or a signal, and configured to detect signals in response to, or derived from, the emitted energy or signal indicating the presence of a blood vessel. The operation of a sensor may be aided by an energy source such as a sensor control device. Suitable ultrasonic systems for blood flow detection include the MedaSonics® CardioBeat® Blood Flow Doppler with Integrated Speaker (Cooper Surgical, Inc., Trumbull Conn. 06611)). Other commercially available Doppler ultrasound sensors suitable for use in the present invention include the Koven model ES 100X MiniDop VRP-8 probe (St. Louis, Mo.) and the DWUNeuro Scan Medical Systems' Multi-Dop B+ system (Sterling, Va.).

As best shown in FIG. 4, the staple deployment mechanisms are oriented at an angle with respect to the plane in which the handles 24 and 26 lie. The angulation provides a more direct attack angle on the uterine arteries to facilitate insertion of the staples through the wall of the patient's fornix and direct the legs into the patient's uterine tissue with the uterine artery disposed between the legs and traversing the occlusion bar. A variety of suitable angulations of the staple deployment mechanism may be provided depending upon patient anatomy.

Closure of a blood vessel, which may be partial or total, is effected by pressure applied through the wall of the patient's vaginal fornix by the occluding bar of the staple. Sufficient pressure or force is applied to the tissue of the vaginal wall by the occluding bar to compress and to at least partially occlude the underlying uterine artery. The blood flow sensor for detecting or locating the uterine artery should be disposed on the leading surface of the staple deployment mechanism and generally should be perpendicular to the tissue-contacting surface of the bar.

FIG. 13 schematically illustrates in part a human female reproductive system, including a uterus 40, uterine artery 41 and vaginal fornix 42. A method of using the uterine artery staple 10 embodying features of the invention includes introducing the staple deployment system into the patient's vaginal canal and advancing the delivery system therein until the leading edges 38 of the staple deployment mechanism 32 is adjacent to the patient's uterine cervix. The staple driver 34 is actuated to press the staple 10 against the wall of the patient's vaginal fornix 42. The position of the handles 12 and 16 are adjusted by the finger grips 24 and 28 which extend out of the patient's vaginal opening to decrease the spacing between the staple deployment mechanisms 25 and 29 and further press the leading edge 38 of the deployment mechanisms against the vaginal fornix 42. With the guidance of the Doppler blood flow sensor 36, the staple deployment mechanism 25 is positioned as close as possible to the patient's uterine artery 41 with the occluding bar 11 of the staple 10 oriented transverse to the uterine artery. When the staple 10 is driven out of the slot 33 into the adjacent tissue of the patient, sufficient pressure is applied to the tissue by the occlusion bar 11 of the staple 10 to facilitate occlusion of the uterine artery 42. The handles 23 and 27 of the staple deployment system 21 are locked by ratchet members 30 and 31 to adjust the space between the staple deployment mechanisms 25 and 29 so that they press against the vaginal fornix adjacent both sides of the patient's uterine cervix to facilitate penetration of the legs 12 and 13 of the staple 10 into the wall of the vaginal fornix and into uterine tissue. The clamped position is maintained until the staples 10 are deployed and then the ratchet members 30 and 31 are released to release the deployment mechanism, so the delivery system 21 may then be withdrawn from the patient.

The staple 10 is retained with the occluding bar 11 applying pressure to occlude the uterine artery for about 0.5 to about 48 hours, preferably about 1 to about 24 hours for effective therapeutic treatment of a uterine disorder, e.g. for fibroids, PPH, DUB and the like. Sufficient bioabsorption has occurred by this time so the staple 10 begins to disintegrate at selected locations (16-19) thereby releasing the occluding pressure applied by the occluding bar 11 to the uterine artery 41. Blood flow sensor 36, which is effective to locate uterine artery 41 by detecting blood flow, may also be utilized to monitor blood flow through the uterine artery to detect the onset of occlusion and the reestablishment of blood flow through the artery. A strand 20 may be utilized to aid in removing all of parts of the staple 10.

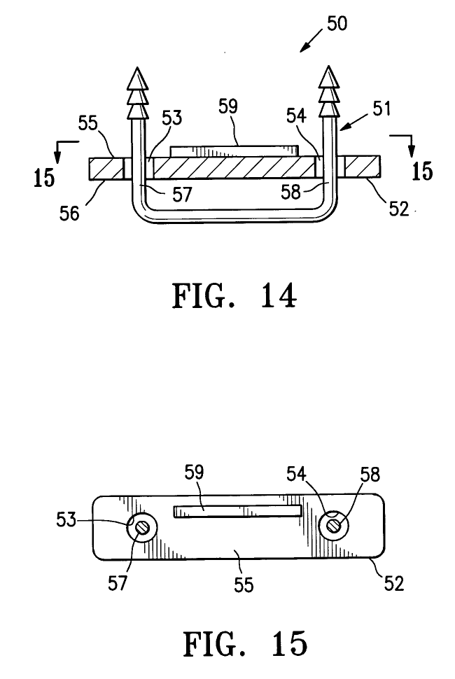

An alternative staple assembly 50 is illustrated in FIGS. 14 and 15 which includes a staple member 51 which is essentially the same as staple 10 as shown in FIG. 1 and a separate occluding bar 52 which has a pair of bores 53 and 54 which extend between the posterior surface 55 and the anterior surface 56. The bores 53 and 54 are configured to slidably receive the legs 57 and 58 of the staple member 51. A blood flow sensor 59 is provided on the posterior surface of the occluding bar 52.

The uterine artery staple embodying features of the invention may be made from any suitable material or combination of materials, including metals such as stainless steel and superelastic shape memory alloys such as nickel-titanium alloys having a stable austenite phase at body temperature, high strength plastics, ceramics, and other materials known in the art. Biocompatible polymers such as polycarbonate, polysulfone, polyester, polyacetal and a variety of fluoropolymers can be suitable for a variety of embodiments of the invention. The device or system may be designed for single use (disposable) or may be sterilizable and capable of multiple use. The bioabsorbable portion of the staple may be formed of bioabsorbable polymeric materials such a PLA, PGA, polycaprolactone, and copolymers of these materials with polyethylene glycol or other hydrophilic agents.

While particular forms of the invention have been illustrated and described, it will be apparent that various modifications can be made to the invention and that individual features shown in one embodiment can be combined with any or all the features of another embodiment described herein. Accordingly, it is not intended that the invention be limited to the specific embodiments illustrated. This invention should be defined by the scope of the appended claims as broadly as the prior art will permit. Terms such as “element”, “member”, “device”, “section”, “portion”, “means”, “steps” and words of similar import when used herein in the appended claims shall not be construed as invoking the provisions of 35 U.S.C. §112(6) unless the claims expressly use the term “means” followed by a particular function without providing specific structure or “step” followed by a particular function without providing specific action.

What is claimed is:

1. A lumen occluding staple which has an occluding bar with a pressure applying surface and which has at least one tissue penetating leg extending distally away from the pressure applying surface of the occlusion bar and having at least one retaining member thereon.

2. The staple of claim 1 wherein the occlusion bar has a tissue penetrating leg extending from an end thereof.

3. The staple of claim 1 wherein the occluding bar and the at least one tissue penetrating legs are separate members and at least one tissue penetrating leg extends through a bore in an end of the occlusion bar.

4. The staple of claim 3 including tissue two penetrating legs.

5. The staple of claim 4 wherein the occluding bar and two tissue penetrating legs are formed into a U-shape.

6. The staple of claim 1 which is formed at least in part of a bioabsorbable material.

7. The staple of claim 6 wherein the bioabsorbable material is selected to be absorbed within a patient's body after a prescribed time period so that the staple will disintegrate and release pressure against an occluded uterine artery.

8. A blood vessel occluding staple which has an occluding bar a pressure applying surface and which has at least one tissue penetating leg extending distally away from the pressure applying surface of the occlusion bar with at least part of the occluding staple being formed of a bioabsorbable material to facilitate separation from penetrated tissue after a predetermined time period to alleviate the pressure applied to tissue by the occlusion bar.

9. A lumen occluding assembly, comprising:
a. an elongated occlusion bar which has a leading edge and a trailing edge and a pressure applying surface on the leading edge and which has first and second bores extending therethrough between the leading and trailing edges; and
b. a staple member which has a pair of penetrating legs disposed within the first and second bores of the occlusion bar and which has a connecting strut extending between proximal ends of the penetrating legs.

10. The lumen occluding assembly of claim 8 including a fluid flow sensor on the leading edge of the occlusion bar.

11. A vascular occlusion system for treating a female patient's uterine disorder, comprising:
a. an elongated handle having an operative proximal end configured to extend out of a patient to facilitate manual manipulation and an operating distal end; and
b. a staple delivery mechanism on the operating distal end of the handle which has a recess for receiving a staple, a driving member in the recess configured to drive the staple out of the recess and into adjacent tissue and which is configured to engage a female patient's vaginal fornix and apply a staple thereto to occlude a uterine artery underlying the patient's vaginal fornix.

12. The vascular occlusion device of claim 11 wherein the driving member is mechanically advanced to deploy the staple.

13. The vascular occlusion device of claim 11 wherein the driving member is hydraulically advanced to deploy the staple.

14. The vascular occlusion device of claim 11 wherein the driving member is electro-mechanically advanced to deploy the staple.

15. The vascular occlusion device of claim 11 wherein a second handle is provided with a manually operative proximal end configured to extend out of the patient during the procedure and a distal end having a staple delivery mechanism which is configured to engaged and apply a staple assembly to the patient's uterine artery and which has a recess for receiving a staple, a driving member on the floor of the recess configured to eject the staple from the recess and drive the staple into the patient's tissue.

16. The vascular occlusion device of claim 15 wherein the first and second handles are rotatably mounted about a pivot point to facilitate adjustment of spacing between the staple delivery mechanisms on the distal ends of the handles.

17. A vaginal staple for occluding a female patient's uterine artery, comprising:
a. an occlusion bar having a pressure applying surface;
b. at least one tissue penetrating staple leg which extends from the pressure applying surface of the occlusion bar and which is configured to restrict removal of the staple leg from tissue; and
c. a portion of the staple leg or occlusion bar which is formed of a bioabsorbable material.

18. The staple of claim 17 wherein two staple legs extend from the pressure applying surface of the occlusion bar.

19. The staple of claim 17 wherein at least part of the staple leg is formed of bioabsorbable material.

20. The staple of claim 19 wherein the bioabsorbable material is a polymer selected from the group consisting of polylactic acid, polyglycolic acid, polycaprolactone and copolymers or blends thereof.

21. A method of treating a female patient's uterine disorder by occluding one or more of the patient's uterine arteries, comprising:
a. providing a staple having one or more legs with tissue retention members,
b. mounting the staple on an intravaginal delivery device;
c. introducing the delivery device and the staple mounted on the distal portion thereof into the patient's vaginal canal and advancing the device therein until the distal portion of the device is adjacent to the patient's vaginal fornix; and
d. pressing the staple mounted on the delivery device against a region of the patient's vaginal fornix and penetrating the tissue with one or more legs of the staple to apply pressure to the tissue to occlude the underlying uterine artery.

22. A lumen occluding staple comprising:
a. an occlusion bar having a posterior pressure applying surface;
b. at least one tissue penetrating staple leg which extends from the posterior pressure applying surface of the occlusion bar and which is configured to restrict removal of the leg from tissue.

23. A lumen occluding staple comprising:
a. an occlusion bar having a posterior pressure applying surface;
b. at least one tissue penetrating staple leg which extends from the posterior pressure applying surface of the occlusion bar; and
c. at least a portion of the occlusion bar or the penetrating staple leg is formed of bioabsorbable material.
